(19) United States
(12) Patent Application Publication
(10) Pub. No.: US 2017/0049466 A1
(43) Pub. Date: Feb. 23, 2017

(54) TISSUE FORCEPS

(71) Applicant: Schmid Healthcare Holdings, LLC, Lubbock, TX (US)

(72) Inventor: Robert P. Schmid, Lubbock, TX (US)

(21) Appl. No.: 14/832,866

(22) Filed: Aug. 21, 2015

(51) Int. Cl. A61B 17/30

(52) U.S. Cl. CPC A61B 17/30

(57) ABSTRACT

A tissue forceps includes a first arm and a second arm. In an unbiased state, the arms extend from a common base along respective lines that form an acute angle resulting in an initial displacement between distal ends of the two arms. The distal end of each arm includes a finger portion that may include a retention notch, a flared footing, or both. Retention notches formed in one or both of the fingers define a retention aperture when the tissue forceps is closed. Retention apertures may be triangular, square, circular or any other suitable shape. The size and shape of the retention aperture may accommodate a surgical needle having a cross section of like size and shape. Flared footings may include inward flares, outward flares, or both. A pair of fingers may include any combination of flared footings combined with any combination of retention notches.

### BACKGROUND

Field of the Disclosure

The present disclosure relates to forceps and, more particularly, tissue forceps for use in medical procedures.

Description of the Related Art

Forceps may be used by doctors and other trained users to grasp or manipulate tissue or surgical needles during surgery or other medical procedures. Adson-Brown forceps are a popular forceps design used by plastic surgeons. These forceps typically include one or more rows of fine teeth on each of two opposing jaws. While the teethed jaws are intended to cause less tissue trauma, they may instead cause undesirable crushing of tissue sufficient to leave behind visible marks.

### BRIEF DESCRIPTION OF THE DRAWINGS

FIG. 1 is an isometric view of a tissue forceps;

FIG. 2 is a side elevation view of the tissue forceps of FIG. 1;

FIG. 3A, FIG. 3B, FIG. 3C, and FIG. 3D illustrate tissue forceps with symmetric retention apertures;

FIG. 4A, FIG. 4B, FIG. 4C, and FIG. 4D illustrate tissue forceps with asymmetric retention apertures; and

FIG. 5A, FIG. 5B, and FIG. 5C illustrate tissue forceps with flared footings.

### DETAILED DESCRIPTION

In the following description, details are set forth by way of example to facilitate discussion of the disclosed subject matter. It should be apparent to a person of ordinary skill in the field, however, that disclosed embodiments are exemplary and not exhaustive of all possible embodiments.

Throughout this disclosure, a hyphenated form of a reference numeral refers to a specific instance of an element and the un-hyphenated form of the reference numeral refers to the element generically or collectively. Thus, widget 12-1 refers to an instance of a widget class, which may be referred to collectively as widgets 12 and any one of which may be referred to generically as a widget 12.

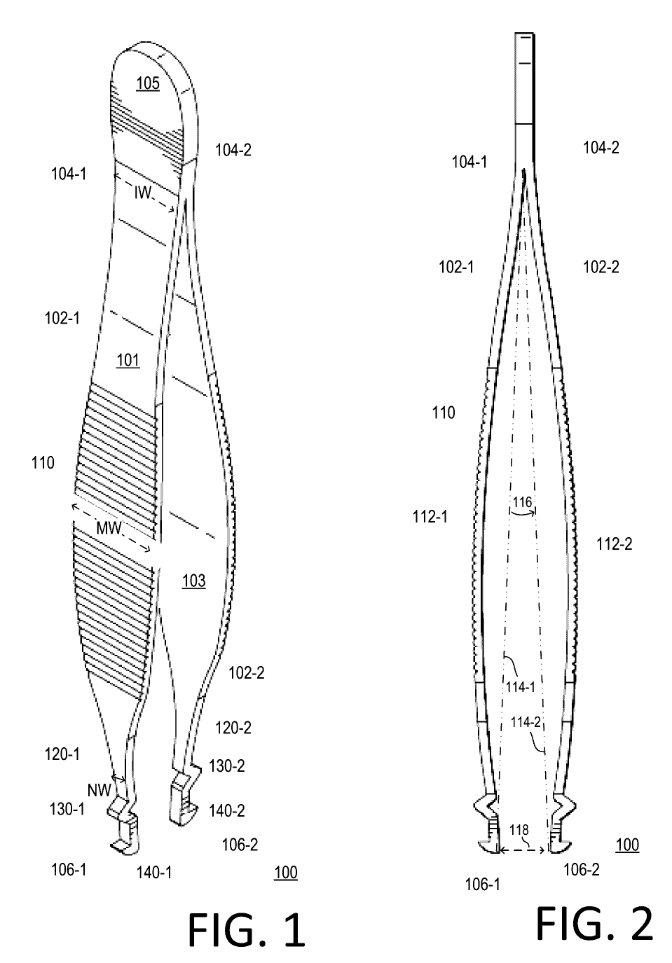

A medical forceps apparatus disclosed herein is suitable for firmly grasping surgical instruments, including surgical needles, and manipulating tissue with little or no tissue damage. The term “forceps” is used herein for both the singular and the plural and is presumptively singular unless it is clear from the applicable context that the plural is intended. As examples: “ . . . tissue forceps 100 may include . . . ” (presumptively singular); “ . . . a plurality of tissue forceps 100 . . . ” (plural). In addition, the term “pair of forceps”, if used, is singular and equivalent to “forceps” (singular). For example, “ . . . In FIG. 1, pair of forceps 100 may include . . . ” (presumptively singular) and “ . . . two or more pairs of forceps 100 . . . ” (plural).

FIG. 1 illustrates a tissue forceps 100 in isometric view and FIG. 2 illustrates tissue forceps 100 in side elevation view. The tissue forceps 100 illustrated in FIG. 1 and FIG. 2 includes a first arm 102-1 having a first end, sometimes referred to herein as the proximal end 104-1, and a second end, sometimes referred to herein as the distal end 106-1, and a second arm 102-2 having a proximal end 104-2 and a distal end 106-2. In some embodiments, proximal end 104-1 of first arm 102-1 can be formed integrally with proximal end 104-2 of second arm 102-2 to form forceps 100. The proximal ends 104 of the arms 102 illustrated in FIG. 1 and FIG. 2 may be welded together or otherwise formed at a common base.

First arm 102-1 and second arm 102-2 may be formed separately or integrally and may be contoured or otherwise formed to facilitate grasping between a thumb and forefinger of a physician, nurse, or other user. The arms 102 of the tissue forceps 100 illustrated in FIG. 1 and FIG. 2 include grooving 110 to aid in such grasping. In at least one embodiment, first arm 102-1 and second arm 102-2 are identical or substantially identical to one another in size, shape, and material. In some embodiments, first arm 102-1 and second arm 102-2 are mirror images or reverse images of one another. Arms 102 may be made of stainless steel, another metal, a suitable plastic or other polymer, or another suitable material.

In at least one embodiment, first arm 102-1 and second arm 102-2 are of the same or substantially the same length. While the length, width, and thickness of each of first arm 102-1 and second arm 102-2 may be of any suitable length, in preferred embodiments, the length of the arms 102 may be in the range between approximately 3 inches (76.2 mm) and approximately 12 inches (304.8 mm). Each of the arms 102 in FIG. 2 is bowed along its length, with its midsection 112 displaced from a line 114 extending between its proximal end 104 and its distal end 106. With no external forces applied to arms 102, forceps 100 may exhibit an initial or unbiased state, illustrated in FIG. 2, in which the arms 102 extend from the common base 105 to their respective distal ends 106 along respective lines 114 that form an acute angle 116 resulting in an initial or unbiased displacement 118 between distal ends 106. In at least one embodiment, the acute angle 116 is in the range between approximately 5 degrees and approximately 25 degrees and the unbiased displacement 118 is in the range between approximately 10 mm and approximately 45 mm.

In at least one embodiment, forceps 100 has sufficient flexibility to transition, under the influence of a closing force, from the unbiased state illustrated in FIG. 2 to a closed state (not depicted) in which distal ends 106 of arms 102 are either in contact with one another or in very close proximity to one another. Forceps 100 may also have sufficient rigidity to return to the unbiased state when the closing force terminates.

Each arm 102 may include or define an obverse side 101 and a reverse side 103. FIG. 1 illustrates arms 102 arranged in an opposing orientation with their respective reverse sides 103 facing one another. The reverse side 103 illustrated in FIG. 1 is smooth or substantially smooth while midsection 112 of obverse side 101 (illustrated in FIG. 2) includes grooving 110 defining a grasping section configured to reduce slipping and improve precise handling by a user while applying a squeezing force to arms 102 to transition distal ends 106 of arms 102 into contact with one another or in close proximity to one another. It will be appreciated that the grasping section may include ridges, raised members, or indented members to reduce slipping and improve precise handling.

Each arm 102 may have a uniform or substantially uniform thickness along its length. In other embodiments, the thickness of either arm 102 may vary smoothly and continuously between its two ends. In at least one embodiment, a minimum thickness of each arm is in the range between approximately 0.5 mm and approximately 2.5 mm and a maximum thickness is in the range between approximately 2.0 mm and 4.0 mm.

A width of either arm 102 may vary along its length line. Each arm 102 illustrated in FIG. 1 has an intermediate width IW at its proximal end 104. The width of each arm 102 increases continuously to a maximum width MW at or near midsection 112 and then tapers continuously to a narrower width NW at distal end 106. In at least one embodiment, the intermediate width IW is in the range between approximately 10 mm and 30 mm, the maximum width MW is in the range between approximately 20 mm and 50 mm, and the narrower width NW is in the range between approximately 5 mm and 25 mm. In at least one embodiment, a displacement between the maximum width MW and the distal end 106 is in the range of 20 mm to 40 mm.

The distal ends 106 of the arms 102 illustrated in FIG. 1 and FIG. 2 include elongated and narrow fingers 120 extending from midsection 112 of each arm 102. A portion of either finger 120 or both fingers 120 may include or define a notch 130 or another type of discontinuity illustrated in FIG. 2, sometimes referred to herein as a retention notch. In at least one embodiment, a displacement between the retention notch and a termination of the finger is in the range between approximately 8 mm and 12 mm.

Each retention notch 130 may define a portion of a retention aperture (not depicted in FIG. 1 or FIG. 2) formed when tissue forceps 100 is biased to a closed position. In some embodiments, both fingers 120 include a notch 130 and the two notches 130 cooperatively define a retention aperture when forceps 100 are biased to a closed position, with first finger 120-1 retention notch 130-1 adjacent to or in close proximity to second finger 120-2 retention notch 130-2.

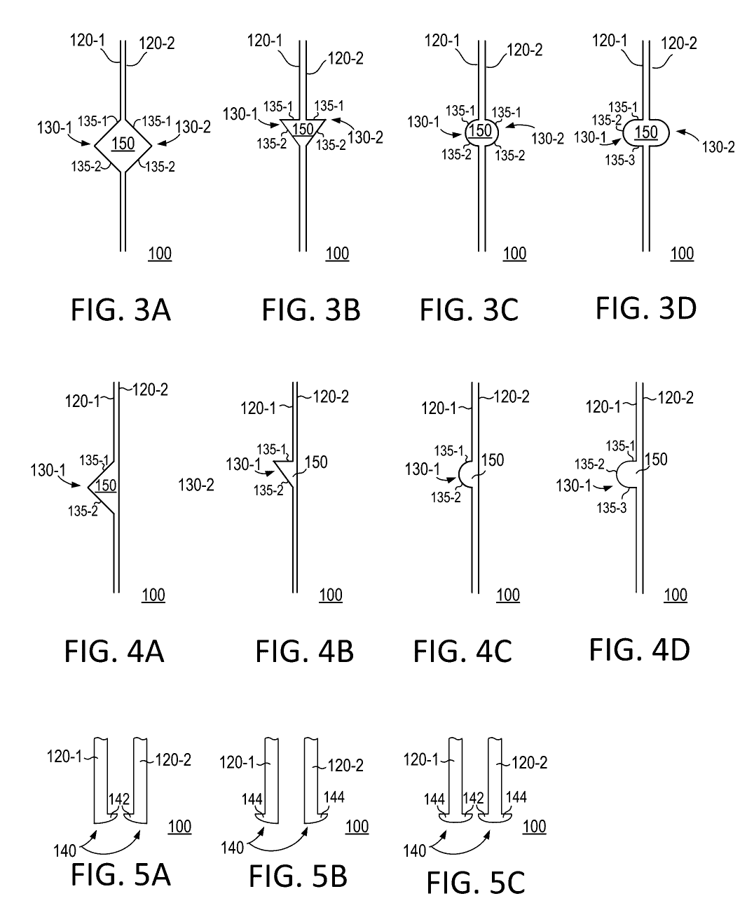

FIGS. 3A, 3B, 3C, and 3D illustrate exemplary configurations for retention notches 130 and retention apertures 150. The outline or shape of each retention aperture 150 reflects or conforms to the cross sectional outline or shape of a needle (not depicted) or another suitable article of surgical hardware. In FIG. 3A through FIG. 3D, each finger 120 includes a corresponding retention notch 130 defined by two or more linear or curved notch segments 135 and each first finger notch 130-1 is a mirror image of the corresponding second finger notch 130-2. In FIG. 3A, each retention notch 130 is a 90 degree corner defined by linear notch segments 135-1 and 135-2 and the pair of retention notches 130-1 and 130-2 define a square retention aperture 150 when the two fingers 120 are brought into close proximity to one another. In other configurations, retention notches 130 may define a retention aperture 150 that comprises a triangle (FIG. 3B), a rectangle (not depicted), pentagon (not depicted), hexagon (not depicted), or another suitable polygon. In other embodiments, each retention notch 130 may include one or more arced or curved notch segments 135 and the notch segments 135 may cooperatively define a retention aperture 150 that comprises a circle (FIG. 3C), a non-circular ellipse (not depicted), or another curved retention aperture. The notch segments 135 illustrated in FIG. 3D include a linear notch segment 135-1, 135-3 and a curved notch segment 135-2 that define a hybrid retention notch 130 and retention aperture 150.

FIGS. 4A, 4B, 4C, and 4D illustrate tissue forceps 100 with asymmetrical retention apertures 150. Each of the illustrated tissue forceps 100 includes a first finger 120-1 that includes a retention notch 130-1 analogous to the first finger retention notches 130-1 illustrated in FIG. 3A through FIG. 3D. However, none of the tissue forceps 100 of FIG. 4A through FIG. 4D have a notch in its second finger 120-2. Other asymmetric retention apertures 150 (not depicted) may include configurations in which both fingers 120 have a retention notch 130, but the two retention notches 130-1 and 130-2 are dissimilar.

Referring to FIG. 5A, FIG. 5B, and FIG. 5C, each finger 120 of tissue forceps 100 may terminate in a flared footing 140. Flared footings 140 may include an inward flare, an outward flare, or a hammerhead flare that includes both an inward flare and an outward flare. The size and configuration of the one or more flared footings 140 may be suitable for adjusting tissue position during surgery or another medical procedure. Additionally, flared footings 140 may be used to grasp the tissue of a patient when first arm 102-1 and second arm 102-2 are brought towards one another through appropriate pressure or squeezing applied by the user.

The flared footings 140 of the fingers 120 illustrated in FIG. 5A include inward flares 142. The flared footings 140 of the fingers 120 illustrated in FIG. 5B include outward flares 144. The flared footings 140 of the fingers 120 illustrated in FIG. 5C include inward flares 142 and outward flares 144. Although the illustrated flared footings 140 include flared footings 140 on both fingers 120, some embodiments may include a flared footing on just one of the two fingers, finger 120-1, for example. Similarly, although the illustrated flared footings 140 employ the same style of flare on both fingers, some embodiments may employ asymmetrically configured flared footings in which the style of flare on the flared footing 140 of finger 120-1 is different than the style of flare on the flared footing 140 of finger 120-2.

The flared footings 140 illustrated in FIG. 5A through FIG. 5C, may be used in combination with the retention apertures 150 illustrated in FIG. 3A through FIG. 3D and FIG. 4A through FIG. 4D. Accordingly, the fingers 120 of a tissue forceps 100 may include any of the retention notches 130 and retention apertures 150 illustrated in or described with respect to FIG. 3A through FIG. 4D and any of the flared footings 140 illustrated in or described with respect to FIG. 5A through 5C.

To the maximum extent allowed by law, the scope of the present disclosure is to be determined by the broadest permissible interpretation of the following claims and their equivalents, and shall not be restricted or limited to the specific embodiments described in the foregoing detailed description.

What is claimed is:

1. A tissue forceps, comprising:
a first arm including:
a first end;
a second end; and
a finger portion, extending from a midpoint to the second end, including at least one added element;
a second arm including:
a first end coupled, at a common base, to the first end of the first arm;
a second end; and
a finger portion extending from a midpoint to the second end;
the first and second arms extending from the common base to their respective second ends along respective lines, wherein, in an unbiased state, the respective lines form an acute angle resulting in an initial displacement between the second ends of the arms and wherein, responsive to a closing force applied to the arms, the arms transition to a closed position with the second end of the first arm in contact with or in close proximity to the second end of the second arm;
wherein the added element includes at least one of:
a retention notch wherein a shape of the retention notch defines, in the closed position, in combination with a finger portion of the second arm, a retention aperture suitable for retaining a surgical needle or a flared footing; and
a flared footing including at least one flare selected from: an inward flare extend towards the second arm and an outward flare extending away from the second arm.

2. The tissue forceps of claim 1, wherein the added element is a retention notch.

3. The tissue forceps of claim 2, where the retention notch includes at least one linear notch segment.

4. The tissue forceps of claim 3, wherein the retention notch defines at least a portion of a polygonal retention aperture.

5. The tissue forceps of claim 4, wherein the polygonal retention aperture comprises an equilateral polygon.

6. The tissue forceps of claim 2, wherein the finger portion of the second arm has no retention notch.

7. The tissue forceps of claim 2, wherein the retention notch includes at least one nonlinear notch segment.

8. The tissue forceps of claim 7, wherein the retention notch defines at least a portion of an elliptical retention aperture.

9. The tissue forceps of claim 2, wherein the retention notch include at least one nonlinear notch segment and at least one linear notched segment.

10. The tissue forceps of claim 1, wherein the added element comprises a first added element and wherein the finger portion of the second arm includes a second added element.

11. The tissue forceps of claim 10, wherein the first added element includes a first retention notch and the second added element includes a second retention notch.

12. The tissue forceps of claim 11, wherein the first retention notch and the second retention notch are of equal size and shape.

13. The tissue forceps of claim 1, wherein the added element includes a flared footing.

14. The tissue forceps of claim 13, wherein the flared footing includes an inward flare.

15. The tissue forceps of claim 13, wherein the flared footing includes an outward flare.

16. The tissue forceps of claim 13, wherein the flared footing includes a hammerhead flare.

17. The tissue forceps of claim 13, wherein the second finger includes a second flared footing.

18. The tissue forceps of claim 17, wherein both flared footings are of the same type of flare.

19. The tissue forceps of claim 1, wherein the at least one added element includes two added elements including a retention notch and a flared footing.

20. The tissue forceps of claim 19, wherein the second arm includes a retention notch and a flared footing.

21. The tissue forceps of claim 1, wherein an outward face of the first arm includes a grooved middle portion.

22. The tissue forceps of claim 1, wherein the first and second arms are bowed and further wherein the midpoint of each arm is displaced from respective lines extending between the first and second ends of the respective arms.
